(19) United States
(12) Patent Application Publication
(10) Pub. No.: US 2007/0062547 A1
(43) Pub. Date: Mar. 22, 2007

(54) Systems for and methods of tissue ablation

(76) Inventor: Carlo Pappone, Milano (IT)

Correspondence Address: Bryan K. Wheelock, Suite 400, 7700 Bonhomme, St. Louis, MO, 63105, US

(21) Appl. No.: 11/478,451

(22) Filed: Jun. 29, 2006

Related U.S. Application Data
(60) Provisional application No. 60/701,225, filed on Jul. 21, 2005.

(51) Int. Cl. A61B 19/00; A61B 18/18

(52) U.S. Cl. 128/898; 606/34; 606/41

(57) ABSTRACT

A method of controlling a remote navigation system that orients the distal end of a medical device in response to user inputs, includes interrupting the operation of the remote navigation system when the user inputs would navigate the medical device to a location where the impedance exceeds a predetermined value. A method of controlling ablation of cardiac tissue to block an errant signal causing an arrhythmia includes ablating tissue until there is a predetermined reduction in the amplitude of the errant signal or a predetermined reduction in local impedance. Controls remote navigation systems can implement these methods.

### CROSS-REFERENCE TO RELATED APPLICATIONS

This application claims the benefit of U.S. Provisional Patent Application Ser. No. 60/701,225, filed Jul. 21, 2005, the entire disclosure of which is incorporated herein by reference.

### BACKGROUND OF THE INVENTION

This invention relates to tissue ablation, and in particular to the control of therapeutic tissue ablation, with remote medical navigation systems for example for the treatment of cardiac arrhythmias.

Tissue ablation in which energy is applied to tissue to destroy it, has a number of important therapeutic applications. One such application is in the treatment of cardiac arrhythmias. A healthy heart typically beats rhythmically and at a predictable rate. However, in some individuals, often those who have underlying heart disease, the heart beats arrhythmically: either too quickly (a condition called tachycardia) or too slowly (a condition called bradycardia). These rhythm abnormalities can occur in the upper chambers of the heart (the atria) or the lower chambers (the ventricles). When the arrhythmia is tachycardia, it is often due to aberrant tissue that is depolarizing and contracting at a faster rate than the sinus node. If the source of the arrhythmia can be identified, ablation can be used to destroy or isolate the tissue that is causing the tachycardia.

Ablation procedures have become a common treatment for arrhythmias and other conditions, but controlling the ablation of tissue remains a challenge. First, it is of course important to control what tissue is ablated and to only ablate tissue that it therapeutically advantageous, and to preserve healthy tissue. Second it is important to control the actual ablation itself to make sure that the ablation is sufficient to achieve the therapeutic goals. Because of these challenges it has been difficult to automate ablation procedures to speed them up and free physicians for less mundane tasks.

### SUMMARY OF THE INVENTION

Some embodiments of the systems and methods of the present invention provide enhanced control over what tissue is ablated, and helps prevent healthy tissue that should not be ablated from being ablated. Some embodiments of the systems and methods of the present invention help improve the quality of the ablation so that the target tissue is not under ablated, and surrounding tissue is not over-ablated. Thus some of the embodiments of the invention facilitate the automation of ablation procedures, for example using a remote medical navigation system.

These and other features and advantages will be in part apparent and in part pointed out hereinafter.

### BRIEF DESCRIPTION OF THE DRAWINGS

FIG. 1 is a flow chart of a method of restricting ablation based on surface impedance in accordance with a first preferred embodiment of the present invention.

FIG. 2A is a flow chart of one implementation of the method of restricting ablation based upon surface impedance in accordance with the first preferred embodiment of the present invention;

FIG. 2B is a flow chart of an alternate implementation of the method of restricting ablation based upon surface impedance in accordance with the first preferred embodiment of the present invention;

FIG. 3A is a flow chart of one implementation of the method of controlling ablation using ECG amplitude in accordance with a second preferred embodiment of the present invention;

FIG. 3B is a flow chart of an alternate implementation of the method of controlling ablation using ECG amplitude in accordance with the second preferred embodiment of the present invention;

FIG. 4A is a flow chart of one implementation of the method of controlling ablation using local impedance in accordance with the second preferred embodiment of the present invention;

FIG. 4B is a flow chart of one implementation of the method of controlling ablation using local impedance in accordance with the second preferred embodiment of the present invention;

FIG. 5 is a diagram of a possible remote navigation system for implementing some of the embodiments of this invention;

FIG. 6 is a sample of an impedance map created in accordance with the principles of some of the embodiments of this invention, on which color indicates impedance of various cardiac surfaces, and regions of high impedance are demarked with dashed lines.

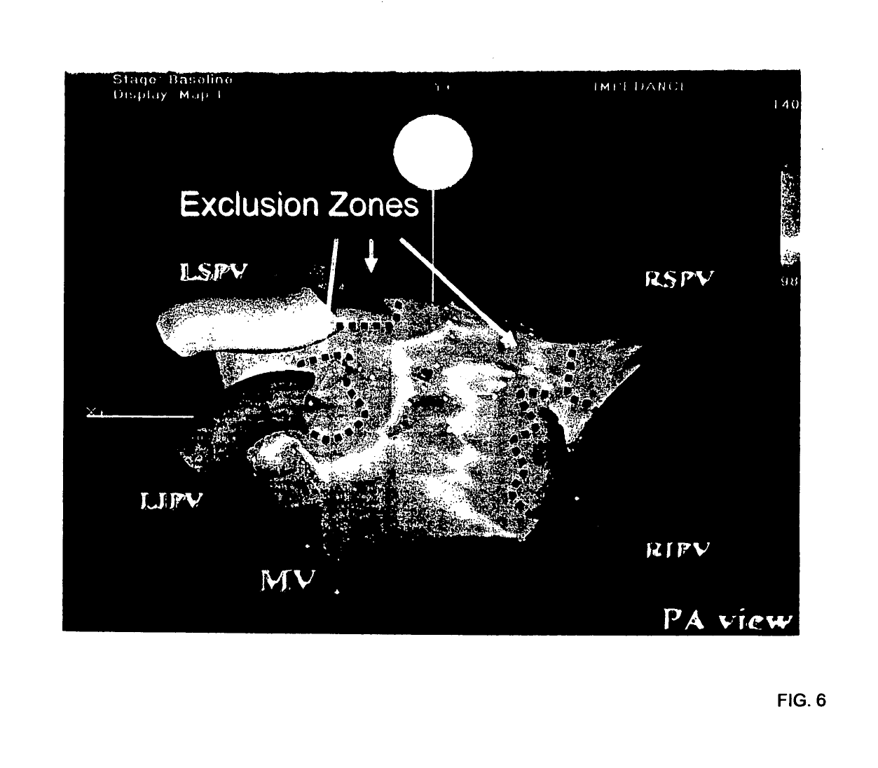

Corresponding reference numerals indicate correspondence parts throughout the several views of the drawings.

### DETAILED DESCRIPTION OF PREFERRED EMBODIMENTS

A first preferred embodiment of a system and method of ablating tissue in accordance with the principles of this invention can improve the control over the location of tissue ablation, and can prevent the ablation healthy tissue that was not intended to be ablated. Different tissues have different basal impedance values, and it is possible to discriminate among tissue types based upon impedance. For example pulmonary veins have a basal impedance that is between about 15 Ω and 20 Ω higher than that of the Left Atrial tissue surface. When ablating cardiac tissue to block conduction paths it would be desirable to avoid ablating the pulmonary veins, as the pulmonary veins tend to stenose after being ablated; this stenosis can lead to permanent injury or death. By mapping the impedance of the surface where the ablation is to take place, “exclusion zones” can be identified where ablation should be prevented or at least avoided.

When using a remote navigation system which orients, and preferably orients and advances a medical device in the body, the systems and methods of this first preferred embodiment can help prevent tissue from being ablated. In a manual mode of operation in which the user submits inputs to the remote navigation system to change the position of the ablation device, a control can be programmed to give the user a warning when the ablation device would be (or alternatively is) positioned on a surface where the impedance exceeds a predetermined value. In one alternative, the controller could actually interrupt the operation of the remote navigation system to prevent the ablation device from being navigated to a position where the impedance exceeds a predetermined value, and give the user an appropriate warning. In a second alternative, the controller could actually interrupt the operation of the ablation system to prevent the ablation of tissue when the ablation device is in a position where the impedance exceeds a predetermined value and give the appropriate warning.

In an automatic mode of operation in which the user identifies one or more locations for ablation, and the system automatically navigates an ablation device to the identified location or locations and ablates the tissue at each location, a control can be programmed to give the user a warning when the user identified a location where the impedance exceeds a predetermined value. In one alternative, the controller could actually interrupt the operation of the remote navigation system to prevent the ablation device from being navigated to a position where the impedance exceeds a predetermined value, and give the user an appropriate warning. In a second alternative, the controller could actually interrupt the operation of the ablation system to prevent the ablation of tissue when the ablation device is in a position where the impedance exceeds a predetermined value, and give the user an appropriate warning.

In embodiments where the device is actually allowed to be navigated, and only the ablation system is interrupted, the device can perform a direct measurement of the local impedance to determine whether it exceeds a predetermined value. However, in most preferred embodiments an impedance map is made of the surface on which the ablations are to take place. This map can be conveniently made with a remote navigation system, which can automatically position a mapping device (which can be the same as the ablation device) in a predetermined network of points, and record the local impedance at each of the points to create a map. Locations between points where the impedance was actually measured can be interpolated. Of course, rather than automatically generate a map, a user can manually navigate the mapping device to a plurality of points on the surface to create a map.

A first preferred embodiment of the methods of this invention is adapted for use with a remote navigation system. The remote navigation system is preferably a mechanical or magnetic navigation system, although the methods could be implemented with any remote navigation system capable of remotely orienting the distal end of medical device, in response to the input of one or more control variables. Mechanical navigation systems typically employ a sleeve or collar for orienting the end of a medical device that telescopes there through. Mechanical elements such as push wires, pull wires, or other devices orient the sleeve or collar. One example of such a device is disclosed in U.S. patent application Ser. No. 10/378,547, filed Mar. 3, 2003, entitled Guide for Medical devices, which is a continuation of Ser. No. 09/875,279, filed Jun. 6, 2001, now U.S. Pat. No. 6,529,761, the disclosures of which are incorporated herein by reference. Magnetic navigation systems typically employ one or more external source magnets for creating a magnetic field in a selected direction which acts upon one or more magnetically responsive elements incorporated into the medical device to orient the distal end of the medical device. Such systems are presently available from Stereotaxis, Inc., St. Louis, Mo.

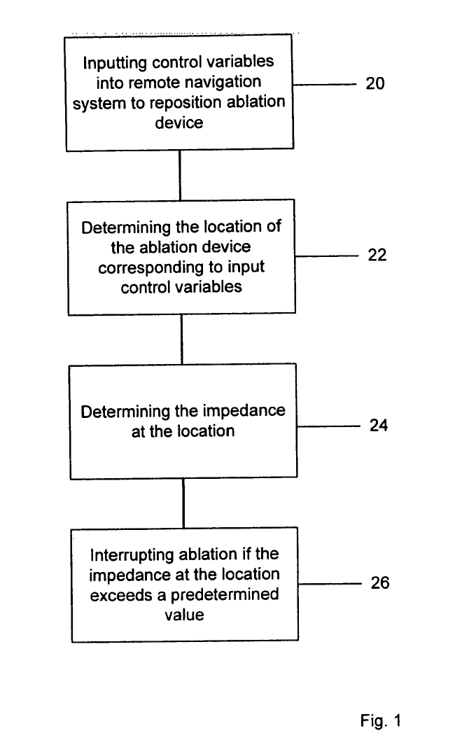

As shown in FIG. 1, at step 20 the user inputs one or more control variables for changing the position of an ablation device, such as an electrophysiology catheter. These can be control variables that are used directly by the remote navigation system, or they can be control variables that are translated for use by the remote navigation system.

At step 22, the new location of the ablation device if the control variables are implemented is determined. This can be done by actually applying the control variables so that the ablation device is moved to the new position, but is more preferably done with a mathematical model which predicts the configuration or position of the device for given control variables. This is disclosed more fully in U.S. patent application Ser. No. 10/448,273, filed May 29, 2003, entitled Remote Control of Medical Devices Using a Virtual Device Interface, and in U.S. patent application Ser. No. 11/170,764, filed Jun. 29, 2005, for Localization of Remotely Navigable Medical Device Using Control variable and Length, which claims priority of U.S. Provisional Patent Application Ser. No. 60/583,855, filed Jun. 29, 2004 (the entire disclosures of which are incorporated herein by reference).

At step 24 the impedance at the new location is determined. If the ablation device is actually navigated to the new location, this can be done simply by sensing with the ablation device. If the ablation device is an RF ablation device, then the ablation electrode can be employed for this purpose. If the ablation device is some other type of ablation device, then an electrode or other sensor can be provided for this purpose. Preferably, however, an impedance map has been prepared. This can be conveniently done with a remote navigation system which can automatically navigate a mapping device to a plurality of locations and sense the impedance at each location. It can also be done by the user manually navigating to a plurality of locations, and sensing the impedance at each manual location. Even where the mapping is automatic, additional points can be manually added as devices are navigated into contact with surfaces during the procedure.

At step 26 the ablation is interrupted if the impedance for the new location exceeds a predetermined value. This can be accomplished in a number of ways. First, the operation of the navigation system can be interrupted to prevent the ablation device from even being navigated to location. As disclosed in U.S. patent application Ser. No. 10/977,466, filed Oct. 29, 2004, entitled Method for Navigating A Remotely Controllable Medical Device Using Pre-Planned Patterns (incorporated herein by reference), the navigation system typically comprises an orientation subsystem and an advancement subsystem. The device can be prevented from reaching the location by interrupting either one or both of the orientation subsystem and advancement subsystem. Another way of interrupting the ablation is to allow the device to be navigated to the new location, but preventing the operation of the ablation device. In the case of an RF ablation device, the RF energy can be interrupted. In the case of other types of ablation devices, their operating power can be similarly interrupted.

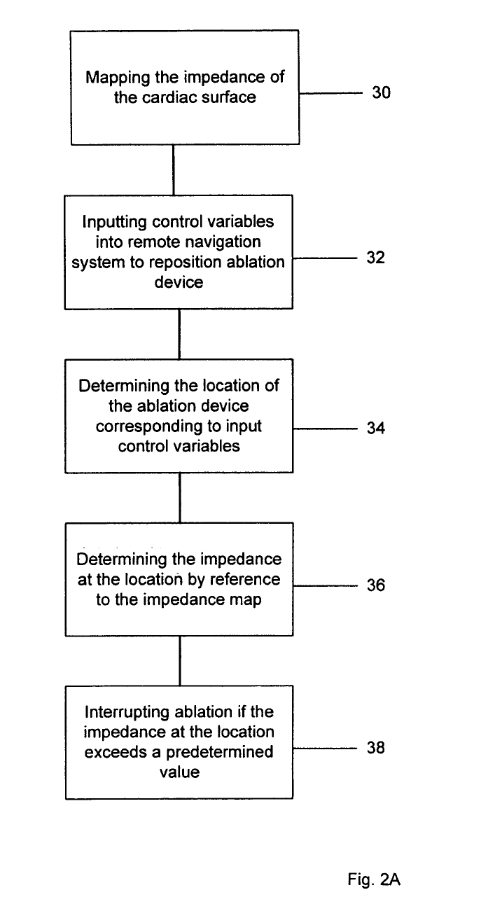

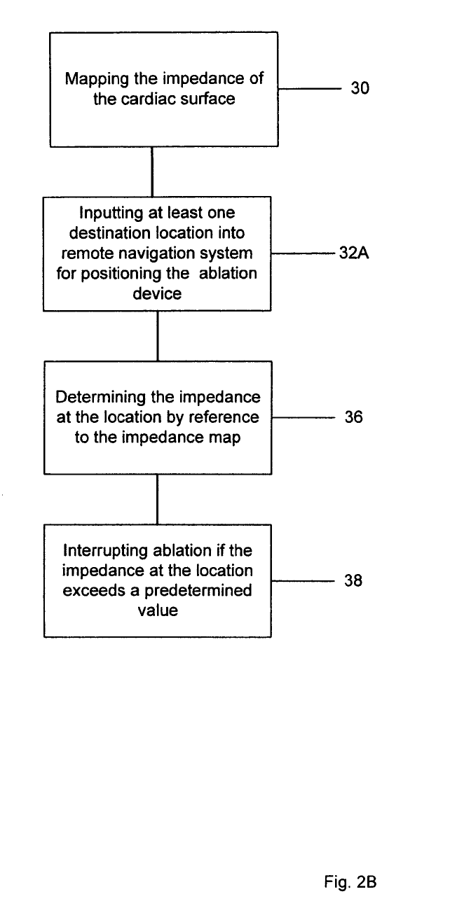

Another version of the first preferred embodiment of the methods of this invention is indicated in FIGS. 2A and 2B. As shown in FIG. 2A, at step 30, the impedance of the surface where the ablations are to take place is mapped. This may be done manually, but is preferably done automatically with a computer-controlled remote navigation system. At step 32 control variables are input. The remote navigation system preferably has a user-friendly interface that facilitates the input of the control variables. At step 34 the new location of the ablation device if the control variables are applied to the remote navigation system is determined. This can be conveniently done with a mathematical model of the device. At step 36, the impedance at the new location is determined with reference to the impedance map created in step 30. Finally, at step 38, ablation is interrupted if the impedance at the new location exceeds a predetermined value (or has some other relationship (e.g., less than or equal to) a predetermined value. The ablation can be interrupted by interrupting the operation of the remote navigation system or the ablation system directly.

As shown in FIG. 2B at step 30, the impedance of the surface where the ablations are to take place is a mapped. This may be done manually, but is preferably done automatically with a computer-controlled remote navigation system. At step 32A a destination is directly input into the remote navigation system. At step 38, ablation is interrupted if the impedance at the new location exceeds a predetermined value (or has some other relationship (e.g., less than or equal to) a predetermined value. At step 36, the impedance at the new location is determined with reference to the impedance map created in step 30. Finally, at step 38, ablation is interrupted if the impedance at the new location exceeds a predetermined value (or has some other relationship, e.g., less than or equal to) a predetermined value. The ablation can be interrupted by interrupting the operation of the remote navigation system or the ablation system directly. Of course, in any of the embodiments instead of interrupting the navigation system or the ablation system, or in addition to interrupting one or both of these systems, a warning indicator can be displayed to warn the user that impedance at the specified location exceeds a predetermined value. This indicator can be a signal light, a symbol on the remote navigation system display, a tactile signal, such as a lock on the navigation system control or on the ablation system control, or anything else that alerts the user to situation.

An override can be provided to allow the user to override the lock out and perform the ablation, if desired.

The methods of the embodiments shown in FIG. 2B can be implemented as part of an automated ablation system where the user identifies a plurality of locations (either manually or with the assistance of a computer) to the remote navigation system at which to ablate, and the system operates automatically to navigate the ablation device to the location and ablate at the location, except locations where the impedance exceeds the predetermined value. When this occurs, as indicated in step 38, ablation is interrupted. The system can either wait for the user to confirm and clear the condition, or the system can move on to the next specified location where ablation is permitted.

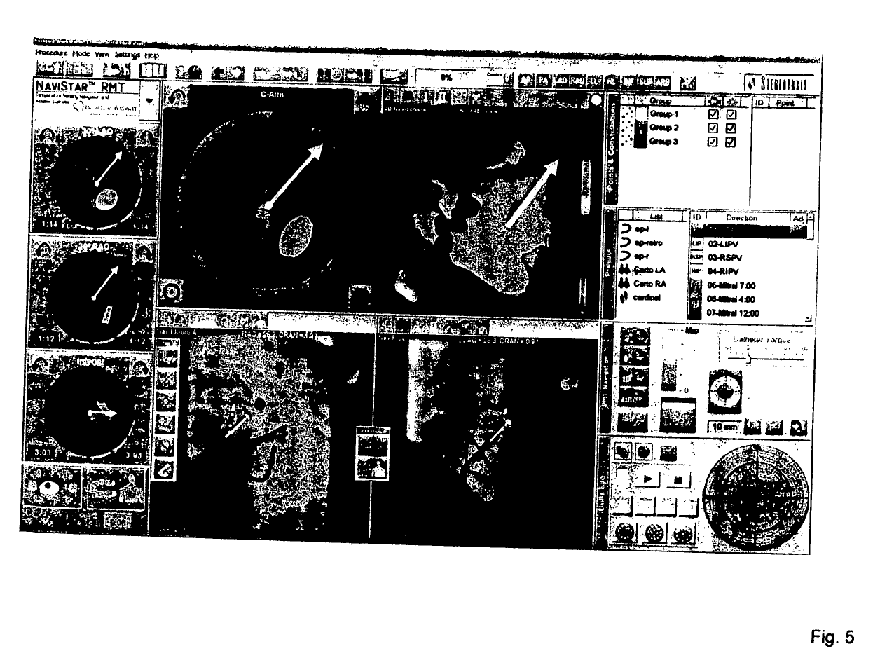

A sample of a user interface for implementing some of the embodiments of this invention is shown in FIG. 5. A sample of an impedance map of a cardiac surface in which local impedance is represented by color. The map is based upon available data points with the values for the areas between points interpolated. Dashed lines indicate boundaries of high impedance that might correspond to the areas where embodiments of the present invention might prevent ablation. Of course, suitable overrides could be provided to allow ablation in appropriate circumstances.

A second preferred embodiment of a system and method of ablating tissue in accordance with the principles of this invention can improve the quality of the ablation of the tissue, making sure that the ablation is complete, but that surrounding healthy tissue is not ablated. Various physiologic changes accompany ablation. For example on a local level, ablation causes edema, which decreases local impedance. This change in local impedance can be measured, and it can be used as a feedback for controlling the ablation. The drop in impedance can be measured on an absolute scale (i.e. a specified drop in resistance, such as 3 Ω), or on a relative scale (i.e. a particular percentage drop in resistance, such as 2% or 3%).

On a broader scale when an ablation is made as part of a line of ablations the ECG signal is affected as each ablation narrows the conduction path for the errant signal. This change in ECG signal can be measured, and it can be used as feed back for controlling the ablation. The drop in ECG amplitude can be measured on an absolute scale (i.e. a specified drop in potential, such as 3 mV), or on a relative scale (i.e. a particular percentage drop in amplitude, such as 20% or 30%). As a typical example, ablation could continue until a 90% drop in peak ECG amplitude is detected.

Each of these measures of ablation effectiveness can be used individually to control the duration of ablation, or they can be used together. Furthermore other measures, for example total time of ablation and/or total energy applied can be combined with one or both of these measures to control the duration of the ablation.

In a manual ablation system, where the ablation is under the direct control of a physician, who, for example, operates a trigger, one or more of these factors (change in local impedance, change in ECG amplitude, time of ablation, and energy applied) can be displayed so that the user can monitor them and stop the ablation. While this can significantly improve manual ablation, further advantages can be obtained in automated ablation. In an automated system ablation can be automatically be terminated when a proper ablation has been completed, for example when the local impedance decreases and/or when the amplitude of the ECG decrease. This helps ensure that the ablation is complete, and it also helps prevent over ablation and damage to surrounding tissue.

Thus in a manual mode the user can trigger an ablation from an ablation device, and one or more indicators can provide information about the extent of the ablation. An indicator can indicate changes in local impedance, an indicator can indicate changes in local ECG amplitude; an indicator can indicate duration of the ablation; and/or an indicator can indicate the total energy applied. The user can continue the ablation until the available indicators indicate that a satisfactory ablation has occurred. In an automated mode the ablation can be initiated automatically and maintained until changes in local impedance and/or changes in local ECG amplitude indicate that satisfactory ablation has occurred. As a fail safe the ablation can also be limited by total duration of ablation or total energy applied or some other factor. In conjunction with a remote medical navigation system, the ablation device can be automatically moved to the next ablation site once the current ablation is completed. Thus a fully automated system for making a plurality of points or a line of points can be provided.

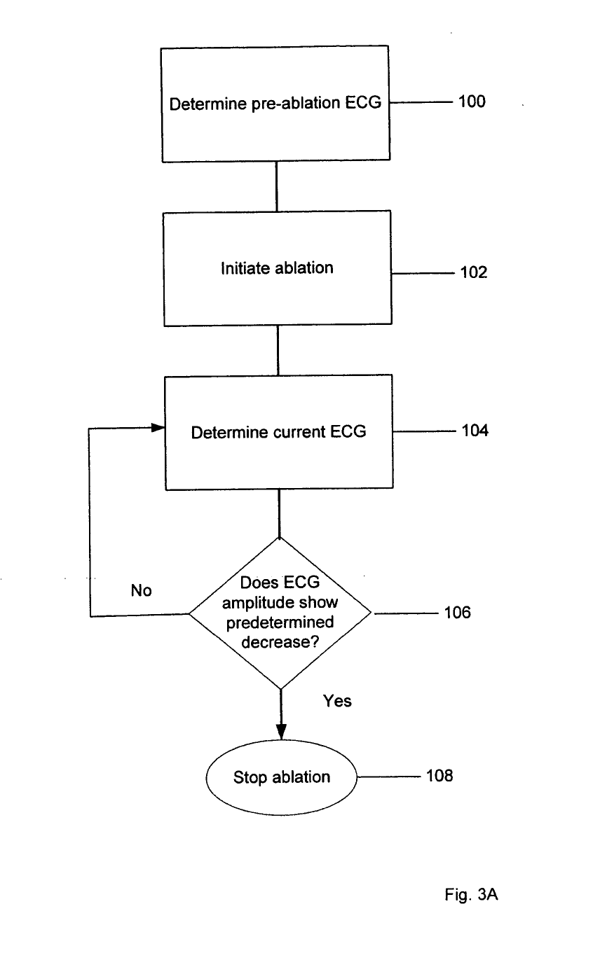

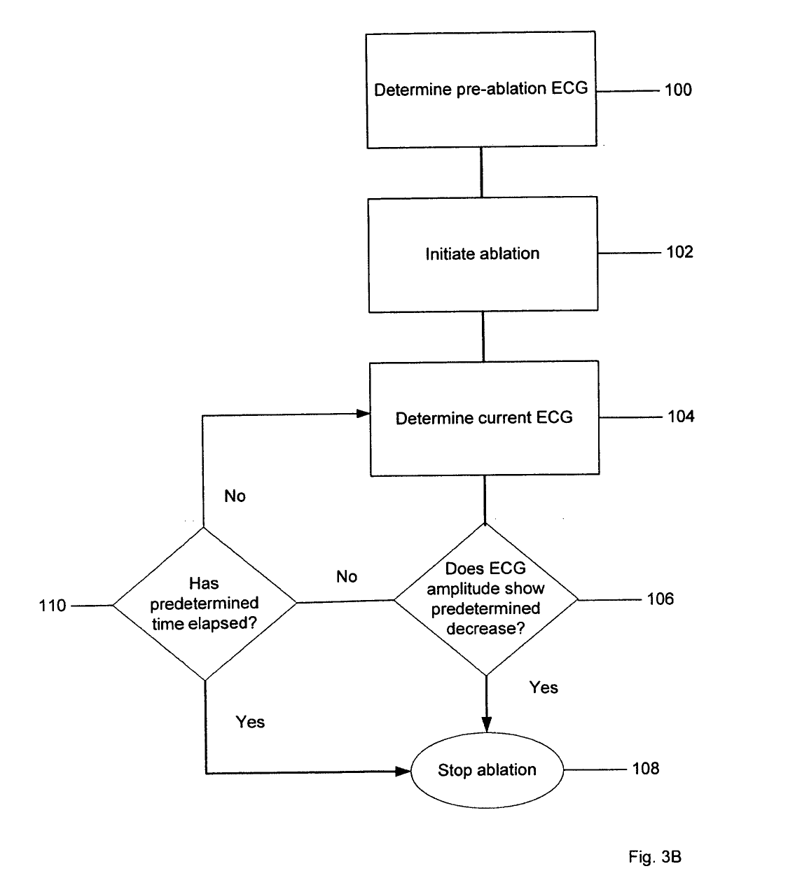

An implementation of a method of controlling ablation is shown in FIGS. 3A and 3B. The method can be used in conjunction with manual navigation, in which the user manually navigates the ablation device to a particular location, and then initiates ablation. The method of control continues the ablation until the ablation is determined to be effective and then stops the ablation. Alternatively, and preferably, the method is used in connection with automatic navigation. The user identified a plurality of locations for ablation, and the method initiates ablation, continues ablation until the ablation is determined to be effect, stops ablation, and allows the remote navigation system to navigate to the next location.

As shown in FIG. 3A, at step 100 the pre-ablation ECG signal is sensed, preferably at least the peak amplitude is stored. At step 102 ablation is initiated. For example RF energy is supplied to an ablation electrode on the distal end of the ablation device. Of course some other mode of ablation could be used, for example laser ablation. At step 104 the current ECG is determined, and at least the peak amplitude is stored. At step 106 the pre-ablation and current ECG are compared. If the amplitude shows a predetermined decrease then at stop 108 the ablation is stopped. If the amplitude does not show a predetermined decrease then ablation continues, and at step 104 the current ECG is sensed. The decrease in amplitude can be an absolute decrease in voltage amplitude, e.g. a 2 mV or 3 mV decrease. Alternatively the decrease in amplitude can be relative decrease in amplitude voltage, e.g. a 30% or a 50% or a 90% decrease in amplitude.

In the alternative shown in FIG. 3B, the steps are the same, but a step has been added to stop the ablation after a predetermined time even if the change in ECG amplitude does not show the predetermined decrease. This prevents over ablation and potential damage to surrounding tissue, if the ablation does not cause the expected effect on ECG signal. Thus if at step 106 the comparison between pre-procedure ECG and current ECG does not show the predetermined decrease, then at step 110, the elapsed time is checked. If the elapsed time exceeds a predetermined value, then ablation is stopped at 108. If the elapsed time has not exceeded the predetermined value, then the ablation continues, and at 104 the current ECG is again determined. Of course, rather than elapsed time some other measure, such as total applied energy, local tissue temperature, or other measure can be used as a limit on the ablation.

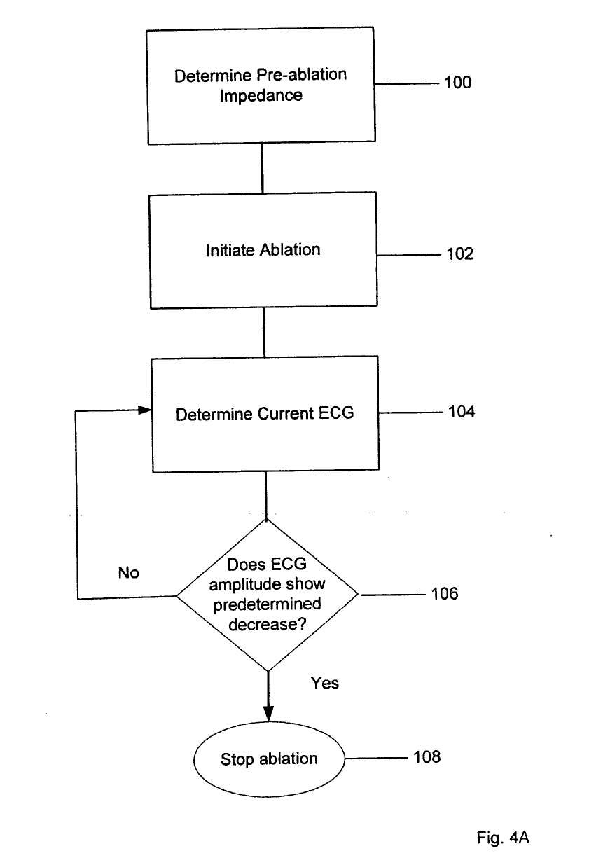

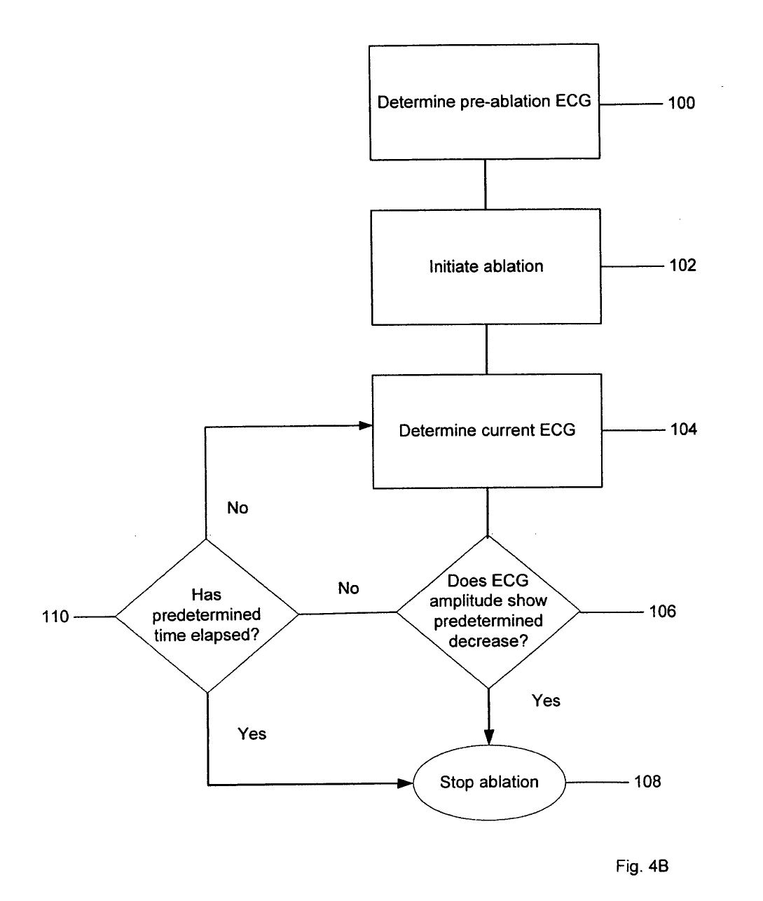

Another implementation of a method of controlling ablation is shown in FIGS. 4A and 4B. The method can be used in conjunction with manual navigation, in which the user manually navigates the ablation device to a particular location, and then initiates ablation. The method of control continues the ablation until the ablation is determined to be effective and then stops the ablation. Alternatively, and preferably, the method is used in connection with automatic navigation. The user identified a plurality of locations for ablation, and the method initiates ablation, continues ablation until the ablation is determined to be effect, stops ablation, and allows the remote navigation system to navigate to the next location.

As shown in FIG. 4A, at step 200 the pre-ablation impedance sensed, and preferably stored. At step 202 ablation is initiated. For example RF energy is supplied to an ablation electrode on the distal end of the ablation device. Of course some other mode of ablation could be used, for example laser ablation. At step 204 the current impedance determined and stored. At step 206 the pre-ablation and current impedances are compared. If the impedance shows a predetermined decrease then at step 208 the ablation is stopped. If the impedance does not show a predetermined decrease then ablation continues, and at step 204 the current impedance is sensed. The decrease in impedance can be an absolute decrease in amplitude voltage, e.g. a 2 Ω or 3 Ω decrease. Alternatively the decrease in amplitude can be relative decrease in impedance, e.g. a 3% or a 5% decrease in impedance.

In the alternative shown in FIG. 4B, the steps are the same, but a step has been added to stop the ablation after a predetermined time even if the change in impedance does not show the predetermined decrease. This prevents over ablation and potential damage to surrounding tissue, if the ablation does not cause the expected effect on impedance. Thus if at step 206 the comparison between pre-procedure impedance and current impedance does not show the predetermined decrease, then at step 110, the elapsed time is checked. If the elapsed time exceeds a predetermined value, then ablation is stopped at 208. If the elapsed time has not exceeded the predetermined value, then the ablation continues, and at 204 the current impedance is again determined. Of course, rather than elapsed time some other measure, such as total applied energy, local tissue temperature, or other measure can be used as a limit on the ablation.

In some embodiments, rather than comparing pre-ablation and current impedance, just the current impedance can be measured and used as a control. In this case, if the impedance reached a particular level, it would indicate satisfactory ablation, and ablation could be discontinued at that location.

1-10. (canceled)

16. A method of ablating tissue on a cardiac surface to treat arrhythmias by blocking errant electrical signals, using an ablation catheter under the control of a remote navigation system, the method comprising:
automatically navigating a catheter to a plurality of locations on the cardiac surface and measuring the impedance at a number of locations to make an impedance map of the surface;
identifying one or more locations on the cardiac surface to ablate and
automatically navigating the ablation element on an ablation catheter to each identified location and energizing the ablation element to ablate tissue at the location, unless the impedance at the location exceeds a predetermined value.

17-19. (canceled)

20. A control for an ablation system comprising a remote navigation system for positioned the ablation element on an ablation catheter on a cardiac surface, and an ablation system for energizing the ablation element to ablate tissue adjacent the ablation element in response to user inputs, the control including an interlock for interrupting operation of at least one of the remote navigation system and the ablation system when the sensed impedance at a point of ablation exceeds a predetermined value.

21. The control according to claim 20 wherein the navigation system includes an orientation controller that orients the distal end of a medical device in an operating region in a subject, and a length controller that extends and retracts the distal end of the medical device, to navigate the distal end of the medical device to a selected destination in response to user inputs.

22. The control according to claim 21 wherein the impedance is determined from an impedance map.

23. The control according to claim 21 wherein the control interrupts only the length controller.

24. The control according to claim 21 wherein the control interrupts only the orientation controller.

25-53. (canceled)

54. A method of controlling ablation of cardiac tissue to block an errant signal causing an arrhythmia, the method comprising ablating tissue until there is a sensing of a predetermined reduction in the amplitude of the errant signal, a predetermined decrease in local impedance, the passage of a predetermined period of time, or the application of a predetermined amount of energy.

55. The method according to claim 54 wherein the predetermined reduction is an absolute reduction in the amplitude of the errant signal.

56. The method according to claim 54 wherein the predetermined reduction is a percentage reduction in the amplitude of the errant signal.

57-59. (canceled)

60. The method according to claim 54 wherein the predetermined reduction is an absolute reduction in the impedance.

61. The method according to claim 54 wherein the predetermined reduction is a percentage reduction in the impedance.

62-65. (canceled)

66. The method according to claim 54 wherein the sensing of a predetermined reduction in the amplitude of the errant signal comprises monitoring the amplitude of the local electrogram; and ablating tissue is performed until a predetermined drop in the amplitude of the local electrogram occurs.

67. The method according to claim 66 where the ablation continues until the first occur of a predetermined drop in the amplitude of the local electrogram or the passage of a predetermined period of time.

68. The method according to claim 66 wherein the ablation continues until the first to occur of a predetermined drop in the amplitude of the local electrogram or the application of a predetermined amount of energy.

69. The method according to claim 66 where the ablation continues until the first occur of a predetermined drop in the amplitude of the local electrogram or a predetermined drop in the local impedance.

70. The method according to claim 66 wherein the ablation continues until the first to occur of a predetermined drop in the amplitude of the local electrogram, a predetermined drop in the local impedance, or the passage of a predetermined amount of time.

71. The method according to claim 66 wherein the ablation continues until the first to occur of a predetermined drop in the amplitude of the local electrogram, a predetermined drop in the local impedance, or the application of a predetermined amount of energy.

72-80. (canceled)

81. A controller for controlling an ablation device to ablate cardiac tissue to block an errant signal causing an arrhythmia, the controller being configured for operating the ablation device to ablate tissue until there is a sensing of a predetermined reduction in the amplitude of the errant signal, a predetermined decrease in local impedance, the passage of a predetermined period of time, or the application of a predetermined amount of energy.

82-89. (canceled)

90. The controller according to claim 81 wherein the predetermined change in impedance of the tissue at the ablation site is a drop in impedance.

91. (canceled)

92. The controller according to claim 81 wherein the predetermined change in impedance of the tissue at the ablation site is a relative drop from the pre-ablation impedance.

93-98. (canceled)
